(19) United States
(12) Patent Application Publication
(10) Pub. No.: US 2007/0134676 A1
(43) Pub. Date: Jun. 14, 2007

(54) Methods and compositions for performing sample heterogeneity corrected comparative genomic hybridization (CGH)

(76) Inventors: Michael T. Barrett, Mountain View, CA (US); Amir Ben-Dor, Bellevue, WA (US); Alicia Scheffer, Redwood City, CA (US); Anya Tsalenko, Chicago, IL (US); Zohar Yakhimi, Ramet Hasharom (IL)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/298,271

(22) Filed: Dec. 8, 2005

(51) Int. Cl. C12Q 1/68; G06F 19/00

(52) U.S. Cl. 435/6; 702/20

(57) ABSTRACT

Methods and compositions for performing sample heterogeneity corrected comparative genomic hybridization (CGH) are provided. In the subject methods, an initial CGH result is processed to account for potential sample heterogeneity to obtain a sample-heterogeneity corrected CGH result. Also provided are methods for evaluating candidate surface-bound nucleic acids, e.g., candidate aCGH probe nucleic acids, to identify probes useful in assaying heterogeneous samples.

### INTRODUCTION

### Background of the Invention

Many genomic and genetic studies are directed to the identification of differences in gene dosage or expression among cell populations for the study and detection of disease. For example, many malignancies involve the gain or loss of DNA sequences resulting in activation of oncogenes or inactivation of tumor suppressor genes. Identification of the genetic events leading to neoplastic transformation and subsequent progression can facilitate efforts to define the biological basis for disease, develop predictors of disease outcomes, improve prognosis of therapeutic response, and permit earlier tumor detection. In addition, perinatal genetic problems frequently result from loss or gain of chromosome segments such as trisomy 21 or the deletion syndromes. Thus, methods of pre and postnatal detection of such abnormalities can be helpful in early diagnosis of disease.

Comparative genomic hybridization (CGH) is one approach that has been employed to detect the presence and identify the location of amplified or deleted sequences. In one implementation of CGH, genomic DNA is isolated from normal reference cells, as well as from test cells (e.g., tumor cells). The two nucleic acids are differentially labeled and then simultaneously hybridized in situ to metaphase chromosomes of a reference cell. Chromosomal regions in the test cells which are at increased or decreased copy number relative to the reference cells can be identified by detecting regions where the ratio of the signals from the two distinguishably labeled nucleic acids is altered. For example, those regions that have been decreased in copy number in the test cells will show relatively lower signal from the test nucleic acids than the reference compared to other regions of the genome. Regions that have been increased in copy number in the test cells will show relatively higher signal from the test nucleic acid.

In a recent variation of the above traditional CGH approach, the immobilized chromosome elements have been replaced with a collection of solid support surface-bound polynucleotides, e.g., an array of BAC (bacterial artificial chromosome) clones or cDNAs. Such approaches offer benefits over immobilized chromosome approaches, including a higher resolution, as defined by the ability of the assay to localize chromosomal alterations to specific areas of the genome.

Genomic lesions associated with developmental disorders are typically present in the germ line and can therefore be detected in samples of homogeneous cell populations. In contrast most tumors arise somatically and the genomic lesions associated with disease are present only in the cells from which the tumor arose (e.g. epithelial cells and adenocarcinoma). Biopsies of somatic tumors typically comprise a mixture of cell types including infiltrating lymphocytes, stromal cells as well as one or more neoplastic cell population(s). Studies have shown that the biological behaviors of surrounding normal cells frequently modify the clinical progress of the tumor cells. Therefore, microarray-based gene expression experiments typically assay the multiple cell populations present in a tumor biopsy. However, non-tumoregenic cells retain a stable diploid genome creating a dilution effect on genomic copy number measures in CGH experiments. Furthermore, multiple neoplastic clones may be present in a single biopsy, where these multiple clones can contain distinct patterns of genomic lesions.

Current routine practice in managing patients with neoplastic lesions includes protocols such as Hemotoxylin and Eosin (H&E) staining to measure the normal cell and tumor cell contents of a biopsy. Typically thresholds for tumor cell versus normal cell ratios are set for selecting biopsies that are suitable for array analyses. Although useful for gene expression and gross measurements of chromosomal alterations, this approach has restricted the resolution of CGH measurements in tumor biopsies. For example the detection of single copy losses and homozygous deletions are particularly susceptible to the presence of even low levels of normal cells in a biopsy sample. Neoplastic cells in many common cancers, such as prostate and breast, frequently present as small tumors and/or as discrete nests of cells surrounded by genomically normal tissues. Thus only a subset of biopsies is sufficiently enriched for tumor cells for array experiments. In addition the use of mixed samples has limited the utility of combined expression and aCGH measures of the same biopsy sample.

One approach for increasing and enriching the number of tumors that can be screened and improving the resolution of aCGH measurements is to purify the neoplastic cells from samples of interest. For example, laser capture microdissection (LCM) can isolate and purify cells of interest based on morphologic differences with surrounding tissues. LCM is widely used in pathology laboratories where routinely processed tissue slides can be used as starting material. The application of LCM has been especially useful for studying neoplastic tissues and biopsies that frequently contain high levels of non-neoplastic cells. A limitation of LCM and related technologies is that even small biopsies frequently contain multiple genetically distinct neoplastic clones that cannot be distinguished by morphology. Clonal populations of cells can be identified and purified for high-resolution somatic analyses by technologies such as fluorescence activated cell sorting (FACS). For example, clonal populations of neoplastic cells often vary in their DNA content (ploidy) and can be identified and purified using DNA specific fluorescent dyes. However, despite their utility in research and clinical applications techniques such as LCM and FACS are currently not widely used in microarray experiments.

To realize the full advantages of aCGH, including the high resolution enabled by an improved probe design process, it is therefore imperative to also provide methods to assess and take into account the cellular composition of interrogated in-vivo samples. In particular it is desirable to account for and to infer, when possible, the components of the mixture of non-neoplastic (e.g. stroma, lymphocytes) and tumor cell populations. In multi-clonal tumor samples this extends to accounting for and inferring, when possible, the various neoplastic clones present in the mixtures and their relative abundance.

The present invention satisfies these and other needs in art. Specifically, this invention addresses the need for experimentally defined computational models to measure copy number variations, such as single copy losses, homozygous deletions, and amplifications in tumor samples containing admixtures of cell populations.

Relevant Literature

United States patents of interest include: U.S. Pat. Nos. 6,465,182; 6,335,167; 6,251,601; 6,210,878; 6,197,501; 6,159,685; 5,965,362; 5,830,645; 5,665,549; 5,447,841 and 5,348,855. Also of interest are published United States Application Serial Nos. 20020006622; 20040241658 and 20040191813, as well as published PCT application WO 99/23256. Articles of interest include: Pollack et al., Proc. Natl. Acad. Sci. (2002) 99: 12963-12968; Wilhelm et al., Cancer Res. (2002) 62: 957-960; Pinkel et al., Nat. Genet. (1998) 20: 207-211; Cai et al., Nat. Biotech. (2002) 20: 393-396; Snijders et al., Nat. Genet. (2001) 29:263-264; Hodgson et al., Nat. Genet. (2001) 29:459-464; and Trask, Nat. Rev. Genet. (2002) 3: 769-778; Zhao et al., Cancer Res., (2004) 64(9): p. 3060-71; Rook, et al., Am. J. Pathol., (2004) 164(1): p. 23-33; Schubert, et al., Am. J. Pathol. (2002) 160(1): p. 73-9; and Barrett, et al., Cancer Res. (2003) 63(14): p. 421 1-7; Barrett et al., Proc. Natl. Acad. Sci. (2004) 101: p. 17765-17770.

### SUMMARY OF THE INVENTION

Methods and compositions for performing sample-heterogeneity corrected comparative genomic hybridization (CGH) are provided. In the subject methods, an initial CGH result is processed to account for potential sample heterogeneity to obtain a sample-heterogeneity corrected CGH result. Also provided are methods for evaluating candidate surface-bound nucleic acids, e.g., candidate aCGH probe nucleic acids, to identify probes useful in assaying heterogeneous samples.

### BRIEF DESCRIPTION OF THE FIGURES

FIG. 1 shows an exemplary substrate carrying an array, such as may be used in the devices of the subject invention.

FIG. 2 shows an enlarged view of a portion of FIG. 1 showing spots or features.

FIG. 3 is an enlarged view of a portion of the substrate of FIG. 1.

### DEFINITIONS

The term “nucleic acid” and “polynucleotide” are used interchangeably herein to describe a polymer of any length composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 100 nucleotides and up to 200 nucleotides in length, or longer, e.g., up to 500 nt in length or longer. However, in representative embodiments, oligonucleotides are synthetic and, in certain embodiments, are under 50 nucleotides in length.

The term “oligomer” is used herein to indicate a chemical entity that contains a plurality of monomers. As used herein, the terms “oligomer” and “polymer” are used interchangeably, as it is generally, although not necessarily, smaller “polymers” that are prepared using the functionalized substrates of the invention, particularly in conjunction with combinatorial chemistry techniques. Examples of oligomers and polymers include polydeoxyribonucleotides (DNA), polyribonucleotides (RNA), other nucleic acids that are C-glycosides of a purine or pyrimidine base, polypeptides (proteins), polysaccharides (starches, or polysugars), and other chemical entities that contain repeating units of like chemical structure.

The term “sample” as used herein relates to a material or mixture of materials, typically, although not necessarily, in fluid form, containing one or more components of interest.

The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The phrase “surface-bound polynucleotide” refers to a polynucleotide that is immobilized on a surface of a solid substrate, where the substrate can have a variety of configurations, e.g., a sheet, bead, or other structure. In certain embodiments, the collections of oligonucleotide target elements employed herein are present on a surface of the same planar support, e.g., in the form of an array.

A “surface-bound polynucleotide with desirable binding characteristics”, as discussed in greater detail below, refers to a surface-bound polynucleotide that has properties that make it suitable for array-based comparative genome hybridization experiments. Such polynucleotides usually exhibit an observed binding behavior that is similar to an expected binding behavior. For example, if binding of a surface-bound polynucleotide to its target sequence is expected to be linear then that polynucleotide is a surface-bound polynucleotide with desirable binding characteristics if it actually exhibits linear binding.

The phrase “labeled population of nucleic acids” refers to mixture of nucleic acids that are detectably labeled, e.g., fluorescently labeled, such that the presence of the nucleic acids can be detected by assessing the presence of the label. A labeled population of nucleic acids is “made from” a chromosome composition, the chromosome composition is usually employed as template for making the population of nucleic acids.

A “non-cellular chromosome composition”, as will be discussed in greater detail below, is a composition of chromosomes synthesized by mixing pre-determined amounts of individual chromosomes. These synthetic compositions can include selected concentrations and ratios of chromosomes that do not naturally occur in a cell, including any cell grown in tissue culture. Non-cellular chromosome compositions may contain more than an entire complement of chromosomes from a cell, and, as such, may include extra copies of one or more chromosomes from that cell. Non-cellular chromosome compositions may also contain less than the entire complement of chromosomes from a cell.

The term “array” encompasses the term “microarray” and refers to an ordered array presented for binding to nucleic acids and the like.

An “array,” includes any two-dimensional or substantially two-dimensional (as well as a three-dimensional) arrangement of spatially addressable regions bearing nucleic acids, particularly oligonucleotides or synthetic mimetics thereof, and the like. Where the arrays are arrays of nucleic acids, the nucleic acids may be adsorbed, physisorbed, chemisorbed, or covalently attached to the arrays at any point or points along the nucleic acid chain.

Any given substrate may carry one, two, four or more arrays disposed on a front surface of the substrate. Depending upon the use, any or all of the arrays may be the same or different from one another and each may contain multiple spots or features. A typical array may contain one or more, including more than two, more than ten, more than one hundred, more than one thousand, more than ten thousand features, or even more than one hundred thousand features, in an area of less than 20 cm² or even less than 10 cm², e.g., less than about 5 cm², including less than about 1 cm², less than about 1 mm², e.g., 100μ², or even smaller. For example, features may have widths (that is, diameter, for a round spot) in the range from 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 mm and less than 1.5 mm, such as more than about 0.8 mm and less than about 1.2 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, the substrate may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulse-jets of either nucleic acid precursor units (such as monomers) in the case of in situ fabrication, or the previously obtained nucleic acid. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used. Inter-feature areas need not be present particularly when the arrays are made by photolithographic methods as described in those patents.

In certain embodiments of particular interest, in situ prepared arrays are employed. In situ prepared oligonucleotide arrays, e.g., nucleic acid arrays, may be characterized by having surface properties of the substrate that differ significantly between the feature and inter-feature areas. Specifically, such arrays may have high surface energy, hydrophilic features and hydrophobic, low surface energy hydrophobic interfeature regions. Whether a given region, e.g., feature or interfeature region, of a substrate has a high or low surface energy can be readily determined by determining the regions “contact angle” with water, as known in the art and further described in copending application Ser. No. 10/449,838, the disclosure of which is herein incorporated by reference. Other features of in situ prepared arrays that make such array formats of particular interest in certain embodiments of the present invention include, but are not limited to: feature density, oligonucleotide density within each feature, feature uniformity, low intra-feature background, low inter-feature background, e.g., due to hydrophobic interfeature regions, fidelity of oligonucleotide elements making up the individual features, array/feature reproducibility, and the like. The above benefits of in situ produced arrays assist in maintaining adequate sensitivity while operating under stringency conditions required to accommodate highly complex samples.

Sensitivity is a term used to refer to the ability of a given assay to detect a given analyte in a sample, e.g., a nucleic acid species of interest. For example, an assay has high sensitivity if it can detect a small concentration of analyte molecules in sample. Conversely, a given assay has low sensitivity if it only detects a large concentration of analyte molecules (i.e., specific solution phase nucleic acids of interest) in sample. A given assay's sensitivity is dependent on a number of parameters, including specificity of the reagents employed (e.g., types of labels, types of binding molecules, etc.), assay conditions employed, detection protocols employed, and the like. In the context of array hybridization assays, such as those of the present invention, sensitivity of a given assay may be dependent upon one or more of: the nature of the surface immobilized nucleic acids, the nature of the hybridization and wash conditions, the nature of the labeling system, the nature of the detection system, etc.

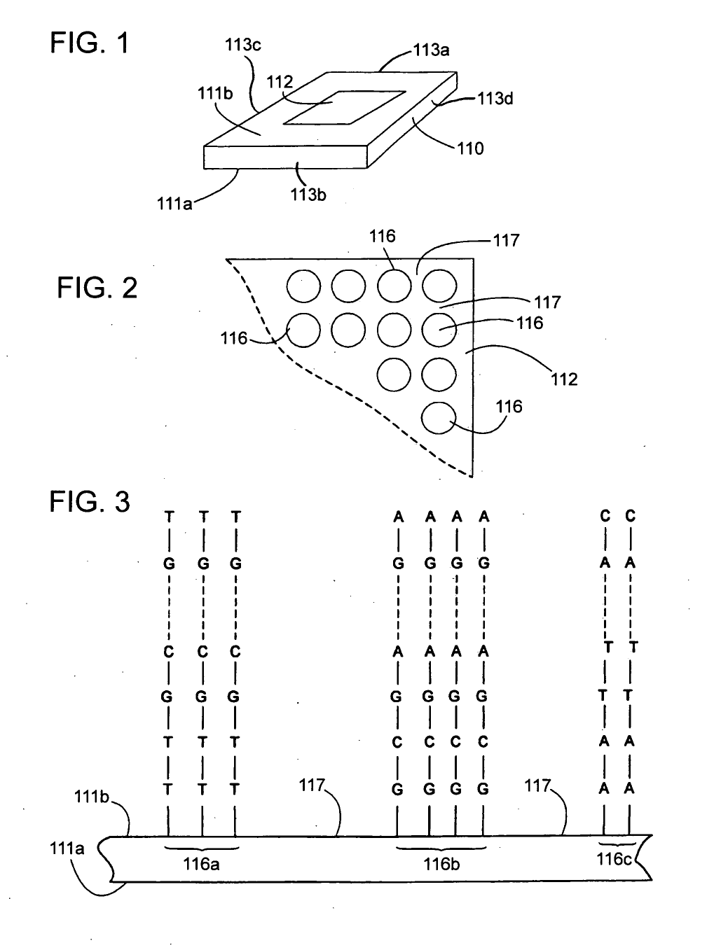

An exemplary array is shown in FIGS. 1-3, where the array shown in this representative embodiment includes a contiguous planar substrate 110 carrying an array 112 disposed on a rear surface 111b of substrate 110. It will be appreciated though, that more than one array (any of which are the same or different) may be present on rear surface 111b, with or without spacing between such arrays. That is, any given substrate may carry one, two, four or more arrays disposed on a front surface of the substrate and depending on the use of the array, any or all of the arrays may be the same or different from one another and each may contain multiple spots or features. The one or more arrays 112 usually cover only a portion of the rear surface 111b, with regions of the rear surface 111b adjacent the opposed sides 113c, 113d and leading end 113a and trailing end 113b of slide 110, not being covered by any array 112. A front surface 111a of the slide 110 does not carry any arrays 112. Each array 112 can be designed for testing against any type of sample, whether a trial sample, reference sample, a combination of them, or a known mixture of biopolymers such as polynucleotides. Substrate 110 may be of any shape, as mentioned above.

As mentioned above, array 112 contains multiple spots or features 116 of oligomers, e.g., in the form of polynucleotides, and specifically oligonucleotides. As mentioned above, all of the features 116 may be different, or some or all could be the same. The interfeature areas 117 could be of various sizes and configurations. Each feature carries a predetermined oligomer such as a predetermined polynucleotide (which includes the possibility of mixtures of polynucleotides). It will be understood that there may be a linker molecule (not shown) of any known types between the rear surface 111b and the first nucleotide.

Substrate 110 may carry on front surface 111a, an identification code, e.g., in the form of bar code (not shown) or the like printed on a substrate in the form of a paper label attached by adhesive or any convenient means. The identification code contains information relating to array 112, where such information may include, but is not limited to, an identification of array 112, i.e., layout information relating to the array(s), etc.

An array is “addressable” when it has multiple regions of different moieties (e.g., different oligonucleotide sequences) such that a region (i.e., a “feature” or “spot” of the array) at a particular predetermined location (i.e., an “address”) on the array will detect a particular sequence. Array features are typically, but need not be, separated by intervening spaces. In the case of an array in the context of the present application, the “population of labeled nucleic acids” will be referenced as a moiety in a mobile phase (typically fluid), to be detected by “surface-bound polynucleotides” which are bound to the substrate at the various regions. These phrases are synonymous with the terms “target” and “probe”, or “probe” and “target”, respectively, as they are used in other publications.

A “scan region” refers to a contiguous (preferably, rectangular) area in which the array spots or features of interest, as defined above, are found or detected. Where fluorescent labels are employed, the scan region is that portion of the total area illuminated from which the resulting fluorescence is detected and recorded. Where other detection protocols are employed, the scan region is that portion of the total area queried from which resulting signal is detected and recorded. For the purposes of this invention and with respect to fluorescent detection embodiments, the scan region includes the entire area of the slide scanned in each pass of the lens, between the first feature of interest, and the last feature of interest, even if there exist intervening areas that lack features of interest.

An “array layout” refers to one or more characteristics of the features, such as feature positioning on the substrate, one or more feature dimensions, and an indication of a moiety at a given location. “Hybridizing” and “binding”, with respect to nucleic acids, are used interchangeably.

The term “stringent assay conditions” as used herein refers to conditions that are compatible to produce binding pairs of nucleic acids, e.g., probes and targets, of sufficient complementarity to provide for the desired level of specificity in the assay while being incompatible to the formation of binding pairs between binding members of insufficient complementarity to provide for the desired specificity. Stringent assay conditions are the summation or combination (totality) of both hybridization and wash conditions.

A “stringent hybridization” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions determines whether a nucleic acid is specifically hybridized to a probe. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C. In instances wherein the nucleic acid molecules are deoxyoligonucleotides (“oligos”), stringent conditions can include washing in 6×SSC/0.05% sodium pyrophosphate at 37° C. (for 14-base oligos), 48° C. (for 17-base oligos), 55° C. (for 20-base oligos), and 60° C. (for 23-base oligos). See Sambrook, Ausubel, or Tijssen (cited below) for detailed descriptions of equivalent hybridization and wash conditions and for reagents and buffers, e.g., SSC buffers and equivalent reagents and conditions.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent hybridization conditions may also include a “prehybridization” of aqueous phase nucleic acids with complexity-reducing nucleic acids to suppress repetitive sequences. For example, certain stringent hybridization conditions include, prior to any hybridization to surface-bound polynucleotides, hybridization with Cot-1 DNA, or the like.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

The term “pre-determined” refers to an element whose identity or composition is known prior to its use. For example, a “pre-determined sample-heterogeneity calibration composition” is a composition containing a known cell type profile, e.g., a known mixture of cell types. An element may be known by name, its function, amount or any other attribute or identifier.

The term “mixture”, as used herein, refers to a combination of elements, that are interspersed and not in any particular order. A mixture is heterogeneous and not spatially separable into its different constituents. Examples of mixtures of elements include a number of different elements that are dissolved in the same aqueous solution, or a number of different elements attached to a solid support at random or in no particular order in which the different elements are not especially distinct. In other words, a mixture is not addressable. To be specific, an array of surface bound polynucleotides, as is commonly known in the art and described below, is not a mixture of capture agents because the species of surface bound polynucleotides are spatially distinct and the array is addressable.

“Isolated” or “purified” generally refers to isolation of a substance (compound, polynucleotide, protein, polypeptide, polypeptide, chromosome, nuclei, cell, etc.) such that the substance comprises the majority percent of the sample in which it resides. Typically in a sample a substantially purified component comprises 50%, preferably 80%-85%, more preferably 90-95% of the sample. Techniques for purifying polynucleotides and polypeptides of interest are well known in the art and include, for example, ion-exchange chromatography, affinity chromatography, flow sorting, and sedimentation according to density.

The terms “assessing” and “evaluating” are used interchangeably to refer to any form of measurement, and includes determining if an element is present or not. The terms “determining,” “measuring,” and “assessing,” and “assaying” are used interchangeably and include both quantitative and qualitative determinations. Assessing may be relative or absolute. “Assessing the presence of” includes determining the amount of something present, as well as determining whether it is present or absent.

The term “using” has its conventional application, and, as such, means employing, e.g. putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

“Contacting” means to bring or put together. As such, a first item is contacted with a second item when the two items are brought or put together, e.g., by touching them to each other.

A “probe” means a nucleic acid that can specifically hybridize to a target nucleic acid, either in solution or as a surface-bound polynucleotide.

The term “validated probe” means a probe that has passed at least one screening or filtering process in which experimental data related to the performance of the probe was used as part of the selection criteria.

“In silico” means those parameters that can be determined without the need to perform any experiments, by using information either calculated de novo or available from public or private databases.

The term “genome” refers to all nucleic acid sequences (coding and non-coding) and elements present in or originating from any virus, single cell (prokaryote and eukaryote) or each cell type and their organelles (e.g. mitochondria) in a metazoan organism. The term genome also applies to any naturally occurring or induced variation of these sequences that may be present in a mutant or disease variant of any virus or cell type. These sequences include, but are not limited to, those involved in the maintenance, replication, segregation, and higher order structures (e.g. folding and compaction of DNA in chromatin and chromosomes), or other functions, if any, of the nucleic acids as well as all the coding regions and their corresponding regulatory elements needed to produce and maintain each particle, cell or cell type in a given organism.

For example, the human genome consists of approximately 3×10⁹ base pairs of DNA organized into distinct chromosomes. The genome of a normal diploid somatic human cell consists of 22 pairs of autosomes (chromosomes 1 to 22) and either chromosomes X and Y (males) or a pair of chromosome Xs (female) for a total of 46 chromosomes. A genome of a cancer cell may contain variable numbers of each chromosome in addition to deletions, rearrangements and amplification of any subchromosomal region or DNA sequence.

By “genomic source” is meant the initial nucleic acids that are used as the original nucleic acid source from which the solution phase nucleic acids are produced, e.g., as a template in the labeled solution phase nucleic acid generation protocols described in greater detail below.

The genomic source may be prepared using any convenient protocol. In many embodiments, the genomic source is prepared by first obtaining a starting composition of genomic DNA, e.g., a nuclear fraction of a cell lysate, where any convenient means for obtaining such a fraction may be employed and numerous protocols for doing so are well known in the art. The genomic source is, in many embodiments of interest, genomic DNA representing the entire genome from a particular organism, tissue or cell type. However, in certain embodiments, the genomic source may comprise a portion of the genome, e.g., one or more specific chromosomes or regions thereof, such as PCR amplified regions produced with specific primers.

A given initial genomic source may be prepared from a subject, for example a plant or an animal, which subject is suspected of being homozygous or heterozygous for a deletion or amplification of a genomic region. In certain embodiments, the average size of the constituent molecules that make up the initial genomic source typically have an average size of at least about 1 Mb, where a representative range of sizes is from about 50 to about 250 Mb or more, while in other embodiments, the sizes may not exceed about 1 Mb, such that they may be about 1 Mb or smaller, e.g., less than about 500 Kb, etc.

In certain embodiments, the subject from which a genomic source is obtained is “mammalian”, where this term is used broadly to describe organisms which are within the class mammalia, including the orders carnivore (e.g., dogs and cats), rodentia (e.g., mice, guinea pigs, and rats), and primates (e.g., humans, chimpanzees, and monkeys), where of particular interest in certain embodiments are human or mouse subjects. In certain embodiments, the genomic source derived from a subject is complex, as the genome of a subject can contain at least about 1×10⁸ base pairs, including at least about 1×10⁹ base pairs, e.g., about 3×10⁹ base pairs.

Where desired, the initial genomic source may be fragmented in the generation protocol, as desired, to produce a fragmented genomic source, where the molecules have a desired average size range, e.g., up to about 10 Kb, such as up to about 1 Kb, where fragmentation may be achieved using any convenient protocol, including but not limited to: mechanical protocols, e.g., sonication, shearing, etc., chemical protocols, e.g., enzyme digestion, etc.

Where desired, the initial genomic source may be amplified as part of the solution phase nucleic acid generation protocol, where the amplification may or may not occur prior to any fragmentation step. In those embodiments where the produced collection of nucleic acids has substantially the same complexity as the initial genomic source from which it is prepared, the amplification step employed is one that does not reduce the complexity, e.g., one that employs a set of random primers, as described below. For example, the initial genomic source may first be amplified in a manner that results in an amplified version of virtually the whole genome, if not the whole genome, before labeling, where the fragmentation, if employed, may be performed pre- or post-amplification.

Array-based CGH (aCGH) assays may be performed in a number of ways. In representative embodiments, the first step is labeling a nucleic acid composition, e.g., a test composition/sample, a calibration composition/sample, a reference composition/sample, etc., to make labeled populations of nucleic acids which may be distinguishably labeled, contacting the labeled populations of nucleic acids with at least one array of surface bound polynucleotides under specific hybridization conditions, and analyzing any data obtained from hybridization of the nucleic acids to the surface bound polynucleotides. Such methods are generally well known in the art (see, e.g., Pinkel et al., Nat. Genet. (1998) 20:207-211; Hodgson et al., Nat. Genet. (2001) 29:459-464; Wilhelm et al., Cancer Res. (2002) 62: 957-960) and, as such, need not be described-herein in any great detail.

Examples of the test and calibration compositions/samples are provided below in the description of the specific embodiments section below. Reference compositions/samples may be made directly from a cell, by isolating a chromosomal extract from the cell. If it is desirable, a reference composition having a composition that is identical to that of a particular cell may be “reconstituted” using isolated chromosomes or other nucleic acids of interest, also called a synthetic reference composition. In certain embodiments, the reference nucleic acid composition is a genomic source from a normal cell, in which the genomic content of the cell is characterized.

In general, the reference composition may contain genomic material from any cell of an organism with a genome e.g., yeast, plants and animals, such as fish, birds, reptiles, amphibians and mammals. In certain embodiments, reference compositions containing genomic material from mice, rabbits, primates, or humans, etc, can be made and used. Suitable cells that may be used as a source of genomic material for use as reference compositions include: monkey kidney cells (COS cells), human embryonic kidney cells (HEK-293, Graham et al. J. Gen Virol. 36:59 (1977)); baby hamster kidney cells (BHK, ATCC CCL 10); chinese hamster ovary-cells (CHO, Urlaub and Chasin, Proc. Natl. Acad. Sci. (USA) 77:4216, (1980); mouse sertoli cells (TM4, Mather, Biol. Reprod. 23:243-251 (1980)); monkey kidney cells (CVI ATCC CCL 70); african green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (hep G2, HB 8065); mouse mammary tumor (MMT 060562, ATCC CCL 51); TRI cells (Mather et al., Annals N.Y. Acad. Sci 383:44-68 (1982)); NIH/3T3 cells (ATCC CRL-1658); and mouse L cells (ATCC CCL-1). Additional cells (e.g. human lymphocytes) and cell lines will become apparent to those of ordinary skill in the art, and a wide variety of cell lines are available from the American Type Culture Collection, 10801 University Boulevard, Manassas, Va. 20110-2209.

In certain embodiments where two or more nucleic acid compositions, such as test and reference compositions are employed, the compositions (or amplification products thereof, are distinguishably labeled using methods that are well known in the art (e.g., primer extension, random-priming, nick translation, etc.; see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). The compositions may be labeled using “distinguishable” labels in that the labels that can be independently detected and measured, even when the labels are mixed. In other words, the amounts of label present (e.g., the amount of fluorescence) for each of the labels are separately determinable, even when the labels are co-located (e.g., in the same tube or in the same duplex molecule or in the same feature of an array). Suitable distinguishable fluorescent label pairs useful in the subject methods include Cy-3 and Cy-5 (Amersham Inc., Piscataway, N.J.), Quasar 570 and Quasar 670 (Biosearch Technology, Novato Calif.), Alexafluor555 and Alexafluor647 (Molecular Probes, Eugene, Oreg.), BODIPY V-1002 and BODIPY V1005 (Molecular Probes, Eugene, Oreg.), POPO-3 and TOTO-3 (Molecular Probes, Eugene, Oreg.), fluorescein and Texas red (Dupont, Boston Mass.) and POPRO3 TOPRO3 (Molecular Probes, Eugene, Oreg.). Further suitable distinguishable detectable labels may be found in Kricka et al. (Ann Clin Biochem. 39:114-29, 2002).

As such, assay protocols of interest include both “one-color” protocols, in which the test and reference samples are all labeled with the same label and assayed separately, e.g., sequentially on the same array or with two different identical arrays, as well as multi-color, e.g., two-color, formats, in which the different samples are distinguishably labeled and contacted with the same array.

The labeling reactions produce a first and second population of labeled nucleic acids that correspond to the test and reference nucleic acid compositions, respectively. After nucleic acid purification and any pre-hybridization steps to suppress repetitive sequences (e.g., hybridization with Cot-1 DNA), the populations of labeled nucleic acids are contacted to an array of surface bound polynucleotides, as discussed above, under conditions such that nucleic acid hybridization to the surface bound polynucleotides can occur, e.g., in a buffer containing 50% formamide, 5×SSC and 1% SDS at 42° C., or in a buffer containing 5×SSC and 1% SDS at 65° C., both with a wash of 0.2×SSC and 0.1% SDS at 65° C.

The labeled nucleic acids can be contacted to the surface bound polynucleotides serially, or, in other embodiments, simultaneously (i.e., the labeled nucleic acids are mixed prior to their contacting with the surface-bound polynucleotides). Depending on how the nucleic acid populations are labeled (e.g., if they are distinguishably or indistinguishably labeled), the populations may be contacted with the same array or different arrays. Where the populations are contacted with different arrays, the different arrays are substantially, if not completely, identical to each other in terms of target feature content and organization.

Standard hybridization techniques (using high stringency hybridization conditions) are used to probe a target nucleic acid array. Suitable methods are described in references describing CGH techniques (Kallioniemi et al., Science 258:818-821 (1992) and WO 93/18186). Several guides to general techniques are available, e.g., Tijssen, Hybridization with Nucleic Acid Probes, Parts I and II (Elsevier, Amsterdam 1993). For a descriptions of techniques suitable for in situ hybridizations see, Gall et al. Meth. Enzymol., 21:470-480 (1981) and Angerer et al. in Genetic Engineering: Principles and Methods Setlow and Hollaender, Eds. Vol 7, pgs 43-65 (plenum Press, New York 1985). See also U.S. Pat. Nos. 6,335,167; 6,197,501; 5,830,645; and 5,665,549; the disclosures of which are herein incorporate by reference.

In representative embodiments, comparative genome hybridization methods comprise the following major steps: (1) immobilization of polynucleotides on a solid support; (2) pre-hybridization treatment to increase accessibility of support-bound polynucleotides and to reduce nonspecific binding; (3) hybridization of a mixture of labeled nucleic acids to the surface-bound nucleic acids, typically under high stringency conditions; (4) post-hybridization washes to remove nucleic acid fragments not bound to the solid support polynucleotides; and (5) detection of the hybridized labeled nucleic acids. The reagents used in each of these steps and their conditions for use vary depending on the particular application.

As indicated above, hybridization is carried out under suitable hybridization conditions, which may vary in stringency as desired. In certain embodiments, highly stringent hybridization conditions may be employed. The term “high stringent hybridization conditions” as used herein refers to conditions that are compatible to produce nucleic acid binding complexes on an array surface between complementary binding members, i.e., between the surface-bound polynucleotides and complementary labeled nucleic acids in a sample. Representative high stringency assay conditions that may be employed in these embodiments are provided above.

The above hybridization step may include agitation of the immobilized polynucleotides and the sample of labeled nucleic acids, where the agitation may be accomplished using any convenient protocol, e.g., shaking, rotating, spinning, pulsing, and the like.

Following hybridization, the array-surface bound polynucleotides are typically washed to remove unbound labeled nucleic acids. Washing may be performed using any convenient washing protocol, where the washing conditions are typically stringent, as described above.

Following hybridization and washing, as described above, the hybridization of the labeled nucleic acids to the targets is then detected using standard techniques so that the surface of immobilized targets, e.g., the array, is read. Reading of the resultant hybridized array may be accomplished by illuminating the array and reading the location and intensity of resulting fluorescence at each feature of the array to detect any binding complexes on the surface of the array. For example, a scanner may be used for this purpose, which is similar to the AGILENT MICROARRAY SCANNER available from Agilent Technologies, Palo Alto, Calif. Other suitable devices and methods are described in U.S. patent application Ser. No. 09/846,125 “Reading Multi-Featured Arrays” by Dorsel et al.; and U.S. Pat. No. 6,406,849, which references are incorporated herein by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). In the case of indirect labeling, subsequent treatment of the array with the appropriate reagents may be employed to enable reading of the array. Some methods of detection, such as surface plasmon resonance, do not require any labeling of nucleic acids, and are suitable for some embodiments.

Results from the reading or evaluating may be raw results (such as fluorescence intensity readings for each feature in one or more color channels) or may be processed results (such as those obtained by subtracting a background measurement, or by rejecting a reading for a feature which is below a predetermined threshold, normalizing the results, and/or forming conclusions based on the pattern read from the array (such as whether or not a particular target sequence may have been present in the sample, or whether or not a pattern indicates a particular condition of an organism from which the sample came).

By “remote location” is meant a location other than the location at which the array is present and hybridization occur. For example, a remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart.

“Communicating” information references transmitting the data representing that information as signals (e.g., electrical, optical, radio signals, etc.) over a suitable communication channel (e.g., a private or public network) and receiving the data. “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

The above description is merely representative of ways of performing CGH to obtain CGH results, and is no way limiting.

### DESCRIPTION OF THE SPECIFIC EMBODIMENTS

Methods and compositions for performing sample heterogeneity corrected comparative genomic hybridization (CGH) are provided. In the subject methods, an initial CGH result is processed to account for potential sample heterogeneity to obtain a sample-heterogeneity corrected CGH result. Also provided are methods for evaluating candidate surface-bound nucleic acids, e.g., candidate aCGH probe nucleic acids, to identify probes useful in assaying heterogeneous samples.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order that is logically possible.

As summarized above, the present invention provides methods for performing sample heterogeneity corrected comparative genomic hybridization (CGH). In addition, the present invention provides methods for evaluating surface-bound nucleic acids for use as probes in CGH assays, particularly for assaying heterogeneous samples. Also provided are compositions for performing these methods, where the methods find use in various applications. In further describing the invention, each of the above methods and compositions for use therein, as well representative applications in which the methods find use, are reviewed in more detail separately below.

Methods of Performing Sample-Heterogeneity Corrected Comparative Genomic Hybridization

The present invention provides methods of performing sample-heterogeneity corrected CGH. By “sample-heterogeneity corrected CGH” is meant that an initial CGH result for a sample is processed to account for potential or known heterogeneity of the sample. Heterogeneity of a sample means that the genomic source used in the CGH assay was obtained from a sample that contained at least two distinct cell types (or other genome containing entity) that have distinct genome content. For example, a genomic source obtained from a tumor biopsy typically contains normal cells (i.e., normal genome copy number) and abnormal cells (i.e., tumor cells with at least one genome copy number variation, or abnormal genome copy number).

In certain embodiments, the initial CGH result for a sample is from a previously-performed CGH assay while in other embodiments a CGH assay is performed on a sample to first obtain an initial result. In either case, the initial CGH result of a sample is provided in a form that is ready to be corrected for potential or known sample heterogeneity. In certain embodiments, the initial CGH assay result is from an array CGH assay performed using the sample of interest. However, the result of virtually any type of initial CGH assay can be processed using the sample-heterogeneity correction method of the present invention.

Processing the initial CGH result to obtain a sample-heterogeneity corrected result can be achieved in several ways. In certain embodiments, processing the initial CGH result involves comparing the initial result to a sample-heterogeneity reference to determine whether the initial sample was heterogeneous and the composition of the heterogeneity. In other embodiments, the cellular heterogeneity of the sample is predetermined by techniques such as H&E staining and flow cytometry and the CGH result is adjusted using an appropriate “sample heterogeneity correction” factor. As will be discussed in greater detail below, a sample-heterogeneity reference can be based on predicted and/or observed variations in CGH results for a CGH probe (or a plurality of probes) that are caused by sample heterogeneity. In these embodiments, the data for at least one probe (or a plurality of probes) in the initial CGH result is compared to the sample-heterogeneity reference for the at least one probe (or a plurality of probes) and a correction factor is assigned to the initial CGH result.

For example, an initial CGH assay of a sample may return a result for a locus of interest (b) that is ambiguous for determining the copy number of the locus of interest (b) in the starting genomic sample. This initial data is then processed by comparing it to a sample-heterogeneity reference and corrected to indicate that the data from locus (b) in the initial CGH assay indicates that the genomic sample was a mixture of cell types that vary at their locus (b) copy number. As such, correction factors of the present invention effectively serve to alter (or change) the result of the initial CGH assay to take into account the potential for, or known contribution of heterogeneity of the initial sample. The changes can take one of a number of forms including annotation of the initial result (e.g., the result indicates that the genomic sample is heterogeneous with regard to locus (b)), correction of the initial result (e.g., the initial result indicates a copy number of X for locus (b) but the processing indicates that the genomic sample was a mixture of cells, one with a copy number of Y for locus (b) and the other with copy number Z at locus (b)), or any other change of an initial result that relates to determining whether the initial sample was heterogeneous.

The Examples section provides specific embodiments of predicting CGH result variations due to sample heterogeneity and of comparing initial CGH results to CGH results previously obtained using samples of known heterogeneity. However, it is to be understood that the specific embodiments in the Examples section are exemplary and are not meant to limit the scope of the algorithms used in correcting CGH results for sample heterogeneity.

In certain embodiments, the sample-heterogeneity corrected CGH results are generated based on predictions of what results would be obtained from CGH assays performed with samples of known heterogeneity. The predicted results are arrived at using an algorithm that extrapolates how a CGH result would appear if the test sample had a specified mixture of cells with different genome copy numbers at a specific locus. For example, the algorithm can predict the CGH result for a particular locus (a) if the test sample contained 90% cells that have the same genome copy number as the reference sample (i.e., a normal copy number) and 10% cells that have twice the normal copy number of locus (a) (i.e., an abnormal copy number, as in a tumor cell). The initial CGH assay result for locus (a) is then compared to the predicted results (also called a sample-heterogeneity reference) and the initial CGH results are corrected to indicate whether the sample is homogenous (i.e., all the cells in the initial sample had the same genomic copy number at locus (a)) or heterogeneous (i.e., the initial sample was a mixture of cells with differing genomic copy number at locus (a)) based on this comparison. The predictive algorithm is used for all or a subset of loci tested in the initial CGH sample to generate a sample-heterogeneity reference that is used to correct initial CGH results for those loci.

In other embodiments, the corrected CGH results are obtained based on observed results from CGH assays performed with heterogeneous samples. These embodiments are similar to the predictive embodiments in that they correct the initial CGH result with regard to the heterogeneity of the sample. However, these embodiments use sample-heterogeneity reference CGH assay results to aid in correcting the initial CGH results for sample heterogeneity. For example, sample-heterogeneity reference CGH assays can be performed on mixtures of two cell types (i.e., X and Y) with known genomic copy number variations at locus (a) (e.g., X has normal copy number at locus (a) and Y has abnormal copy number at locus (a)). Sample-heterogeneity reference CGH results of progressive mixtures of these cells (e.g., 100% X; 90% X and 10% Y; 75% X and 25% Y; etc.) with regard to locus (a) are obtained and analyzed to provide a correction algorithm that can be used to correct CGH assay results for locus (a) performed on samples of unknown heterogeneity. Any number of loci can be corrected using observationally-based embodiments. For example, in embodiments in which the sample-heterogeneity reference CGH assays are array-based CGH assays (aCGH), sample-heterogeneity reference results can be obtained for a plurality of specific genomic probes using a plurality of different sample-heterogeneity reference sample compositions.

The number of data points in an observational sample-heterogeneity reference can be quite large. For example, a CGH array may have as many as one hundred thousand features (e.g., probes) or more and the number of distinct mixed samples analyzed to generate the sample-heterogeneity reference can be from about 2 to about 100, from about 5 to about 60, and including from about 10 to about 30. As such, a sample-heterogeneity reference data set may contain more than ten million data points. Alternatively, a sample-heterogeneity reference can have as few as about one thousand data points or less, including as few as about 100 data points or less, or 10 or fewer data points.

The sample-heterogeneity reference samples used in generating the sample-heterogeneity reference CGH assay results can be made from a variety of genomic sources, including primary cells, cell lines, tumor cells, transgenic cells, or any number of genome-containing biological entities. In addition, the reference samples can be synthetic, meaning that they are made by combining specific genomic components (e.g., chromosomes) or nucleic acids of interest in known copy numbers to the reference sample. In certain embodiments, the precise genomic copy number variation(s) present in the sample-heterogeneity reference sample is known, whereas in other embodiments the precise genome copy number variation is unknown. For example, a sample-heterogeneity reference sample might comprise known mixtures of cells, one of which is from a newly discovered class of tumor that has yet to be fully characterized. However, it is still useful to have sample-heterogeneity reference CGH assays using these tumor cells so that their sample-heterogeneity reference CGH profile can be used to correct CGH assays that may contain similar (or the same) tumor type.

It is to be understood that sample-heterogeneity correction of the initial CGH results can be achieved using any combination of predictive and observational correction methods, as reviewed above. Indeed, in certain embodiments, an initial CGH result is compared to a number of distinct predictive sample-heterogeneity references and/or a number of distinct observational sample-heterogeneity references to correct the results.

The predicted and/or observed sample-heterogeneity reference data can be viewed and utilized in a number of ways. For example, the sample-heterogeneity reference data can be printed on a suitable substrate (e.g., paper) and an individual can process initial CGH results to obtain corrected CGH results using the data. Alternatively, the sample-heterogeneity reference data can be stored in a database, including a computer database, which is accessed and used to process the initial CGH result to obtain a corrected CGH result. In a further embodiment, an initial CGH result can be submitted (e.g., transmitted) by a client to a vendor who then processes the initial CGH results according to the methods of the present invention and transmits the corrected results to the client. As another example, a client can send a sample to a vendor who then performs a CGH assay using the sample and processes the results according to the methods of the present invention and then transmits the corrected results to the client.

In addition, correction of the data can be presented in many ways. For example, the corrected result can be presented as a global assessment of the heterogeneity of the sample (e.g., the sample is homogenous, the sample heterogeneous, or the sample is heterogeneous with X % of a cell type with genomic profile A and Y % of a cell type with genomic profile B). Alternatively, the correction of the data can be in the form of annotations to the results returned for specific probes (e.g., based on the results of probes A, B, C this sample is heterogeneous). Further the correction can provide analysis of whether variation in a probe is likely due to sample heterogeneity or to variations in the CGH assay itself (e.g., while the sample binding to probe A indicates sample heterogeneity, the sample binding to probes C through Q indicate that the variation in probe A binding is due to variation in performing the CGH assay rather than to sample heterogeneity). This is just a sample of the types of corrected results and is in no way meant to limit the results of the sample heterogeneity correction methods of the subject invention.

Computer-Related Embodiments

The invention also provides a variety of computer-related embodiments. Specifically, the data analysis methods described in the previous section may be performed using a computer. Accordingly, the invention provides a computer-based system for analyzing data produced using the above methods in order to correct initial CGH results with regard to sample heterogeneity.

In certain embodiments, the CGH correction methods are coded onto a computer-readable medium in the form of “programming”, where the term “computer readable medium” as used herein refers to any storage or transmission medium that participates in providing instructions and/or data to a computer for execution and/or processing. Examples of storage media include floppy disks, magnetic tape, CD-ROM, a hard disk drive, a ROM or integrated circuit, a magneto-optical disk, or a computer readable card such as a PCMCIA card and the like, whether or not such devices are internal or external to the computer. A file containing information may be “stored” on computer readable medium, where “storing” means recording information such that it is accessible and retrievable at a later date by a computer.

With respect to computer readable media, “permanent memory” refers to memory that is permanent. Permanent memory is not erased by termination of the electrical supply to a computer or processor. Computer hard-drive ROM (i.e. ROM not used as virtual memory), CD-ROM, floppy disk and DVD are all examples of permanent memory. Random Access Memory (RAM) is an example of non-permanent memory. A file in permanent memory may be editable and re-writable.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” references any hardware and/or software combination that will perform the functions required of it. For example, any processor herein may be a programmable digital microprocessor such as available in the form of a electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

In certain embodiments, the subject methods include a step of transmitting data or results from at least one sample-heterogeneity corrected CGH result, as described above, to a remote location.

Utility

The above-described methods find use in any application in which one wishes to perform CGH using a test sample that is known to be or could potentially be heterogeneous (e.g., a mixture of cells). In addition, the subject methods can be used to improve the detection of copy number aberrations in mixed samples with CGH probes and arrays that were not designed with sample heterogeneity processing. In all cases, the subject methods enable the use of CGH measurements for a variety of applications where clonal variation and cellular admixtures occur in the sample(s).

In a representative embodiment, the sample-heterogeneity correction methods of the invention find use in diagnosing a subject for a physiological condition associated with an abnormal genomic copy number at a locus of interest. In many of these embodiments, the physiological condition is a disease condition, including a wide variety of congenital or neoplastic disease conditions.

For example, the subject methods are useful in analyzing tumor biopsies from subjects. As discussed in previous sections, tumor biopsies typically comprise both normal and abnormal cells (in a known or unknown ratio). In representative embodiments, the application of the sample-heterogeneity correction methods will enable accurate CGH measurements of individual cell populations (e.g. tumor cells) in biopsies containing cell mixtures (e.g. normal and tumor cells). Furthermore, by employing the sample-heterogeneity correction methods of this invention, it is possible to determine whether the biopsy comprises cells with genome copy number variations (e.g., tumor cells) without having to first separate the normal cells from the abnormal cells, which is typically difficult and often not possible (e.g., when no method exists to isolate the normal and abnormal cells in the particular biopsy sample). The present sample-heterogeneity correction method would not only indicate whether the biopsy sample comprises cells with genome copy number variations (e.g., a tumor cell), but can also provide valuable diagnostic data as to the type of tumor present in the biopsy and its relative abundance in the biopsy sample. This information would greatly improve current diagnostic techniques and lead to more timely therapeutic intervention, thereby benefiting patients. As such, determining that a tumor biopsy contained a specific sub-population of tumor cells (by virtue of the specific genomic variation identified) will provide an indication as to which therapy might be most effective.

In addition, the subject invention is useful in monitoring the responses and behaviors of tumor cell clones in patient samples before, during and after therapy. Clonal markers, such as somatic mutations, DNA ploidy, and translocation breakpoints determined at the initiation of treatment could be used to provide a measure of the tumor cell heterogeneity. Subsequent CGH assays could then use the correction processing of the subject invention to follow the behavior and pattern of clones as they emerge, evolve, or are eliminated from the tumor tissue over time.

In related embodiments, the methods of the subject invention can be used to model the emergence of drug resistance of tumor cells in vitro or in vivo. For example, a population of tumor cells (or a tumor cell clone) can be harvested and grown in the presence of a therapeutic agent. During the course of the culture, a portion of the tumor cells can be processed according to the methods of the subject invention to determine its clonal composition. If/when tumor clones emerge that are resistant to the therapeutic agent of interest, the process can be repeated (either on individual resistant clones or on the resistant population) using a different drug (or drug combination).

The subject sample-heterogeneity correction methods are also useful in quality control applications for clinical, preclinical and basic research in which an analysis of sample integrity is necessary for acquisition of reliable data. For example, implementation of the subject sample-heterogeneity correction methods will detect whether the reference sample (the sample to which the test sample is compared) is homogenous and thus appropriate for use in CGH assays. In addition, the subject sample-heterogeneity correction methods can detect when a test sample that is thought to be homogenous is contaminated with cells of a differing genomic makeup.

Kits

Also provided are kits for use in the subject invention, where such kits may comprise containers, each with one or more of the various reagents/compositions utilized in the methods, where such reagents/compositions typically at least include at least one probe (e.g., oligonucleotide) identified according to the methods of the invention. In addition, the kits may include a sample-heterogeneity reference recorded on a substrate. In certain embodiments, a plurality of probes are provided that are immobilized on a substrate in the form of an array, e.g., one or more arrays of oligonucleotide features. In addition, reagents employed in labeled nucleic acid production, e.g., random primers, buffers, the appropriate nucleotide triphosphates (e.g. dATP, dCTP, dGTP, dTTP), DNA polymerase, labeling reagents, e.g., labeled nucleotides, and the like are also provided. Where the kits are specifically designed for use in aCGH applications, the kits may further include labeling reagents for making two or more collections of distinguishably labeled nucleic acids according to the subject methods, an array of features, hybridization solution, etc.

Finally, the kits may further include instructions for using the kit components in the subject methods. The instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette, etc.

Methods of Evaluating a Probe for Use in CGH

The present invention also provides methods and compositions for evaluating a candidate probe for suitability for use in a CGH assay. In brief, these embodiments of the invention include the following steps: (a) performing a CGH assay with a candidate probe on a plurality of different sample-heterogeneity calibration compositions to obtain a plurality of CGH results for the candidate probe; and (b) evaluating the plurality of CGH results to assess the candidate CGH probe nucleic acid, e.g., for suitability in a CGH assay. In further describing these aspects of the present invention, calibration and nucleic acid compositions are described first, followed by a more in-depth description of the subject methods of this embodiment. Finally, representative kits and computer programming for use in practicing the subject methods are reviewed in greater detail.

Calibration Compositions

In general, sample-heterogeneity calibration compositions comprise a known composition of cells. By known composition of cells means that the identity of the cells is known and the genome copy number for at least one genomic locus is known for the cells. In certain embodiments, the genome copy number of the cells in the sample-heterogeneity calibration composition is known for most or all loci of the genome. The sample-heterogeneity calibration compositions can contain a number of different cell types, including from about 1 to about 10 cell types, often from about 1 to about 5, and including from about 2 to about 3 cell types.

In a representative embodiment, a plurality of sample-heterogeneity calibration compositions having varying mixtures of two cell types are employed in the probe evaluation methods. In certain of these embodiments, the first cell type contains a normal genome copy number (e.g., is a normal cell) and the second cell type contains an abnormal genome copy number of at least one genomic locus (or multiple loci). These two cell types are mixed in progressively increasing ratios of the first to the second cell type to generate the plurality of sample-heterogeneity calibration compositions. As such, the sample-heterogeneity calibration compositions include samples that have 100% of the first cell type, 100% of the second cell type, and a number samples in which the ratios of the first cell type to the second cell type ranges from about 0.1% to about 99.9%. Depending on the embodiment, the number of sample-heterogeneity calibration samples used in the probe evaluation methods of the present invention includes from about 2 to about 100, from about 5 to about 60, and including from about 10 to about 30.

In certain embodiments, sample-heterogeneity calibration compositions may be made directly from a cell, by isolating a chromosomal extract from the cell. If it is desirable, a sample-heterogeneity calibration composition having a desired composition may be generated using isolated chromosomes or nucleic acids of particular interest, also called a synthetic calibration composition.

Methods

As stated above, the present invention provides methods and compositions for evaluating a candidate probe for suitability for use in a CGH assay. As such, the present invention provides methods for identifying surface-bound nucleic acids (e.g., oligonucleotides) that are suitable for use in aCGH. In brief, these embodiments of the invention include the following steps: (a) performing a CGH assay with a candidate probe on a plurality of different sample-heterogeneity calibration compositions to obtain a plurality of CGH results for the candidate probe; and (b) evaluating the plurality of CGH results to assess the candidate CGH probe nucleic acid, e.g., for suitability in a CGH assay.

In certain embodiments, the aCGH assay is performed using sample-heterogeneity calibration compositions (as described above) that comprise mixtures of cell types in which one cell type has a genomic copy number variation at a locus to which a candidate probe is predicted (or designed) to bind in an aCGH assay. In this way, the binding characteristics of the probe can be evaluated under conditions that mimic an unknown sample comprising a mixture of cells at least one of which has a genome copy number variation at the locus of interest. Conversely, the sample-heterogeneity calibration compositions comprise cells with a genomic copy number variation at a locus different than the locus to which a candidate probe is predicted (or designed) to bind in an aCGH assay. In this case, alterations in the sample heterogeneity should not affect the binding characteristics of the probe.

In certain embodiments, the evaluating step comprises comparing empirical results for at least some of the calibration compositions to expected results for the calibration compositions. For example, a set of candidate probes are used in aCGH assays using the calibration compositions described above. Because the genome content and heterogeneity of the calibration sample are known, the binding characteristics of the candidate probe is predictable using the predictive methods for sample-heterogeneity correcting detailed in the previous section and the Experimental section below (i.e., using a predictive algorithm). If, upon evaluation of the aCGH data, the predicted (or expected) results correlate with the empirical results obtained for a probe, then the probe is a viable candidate for use in aCGH assays in general, and in the methods of the subject invention in particular. In certain embodiments, the predicted results are based on a linear relationship between the copy number of a probe's respective target in the sample and the binding characteristics observed for that probe. In other words, a two-fold increase in the copy number of a target from locus (a) in a CGH sample should result in a two-fold increase in the detected binding of that target to a probe specific for locus (a). In certain embodiments, if a give probe demonstrates this linear behavior, it is determined to be acceptable.

By providing a method of assessing surface-bound polynucleotides for their suitability for use in the sample-heterogeneity correction aCGH methods described above, candidate surface-bound polynucleotides (e.g., oligonucleotides) may be screened to identify surface-bound polynucleotides with desirable binding characteristics. The sequence of a candidate aCGH probes can be done in silico using any number of probe design methods known to those of skill in the art. In this way, candidate probes can be chosen that meet some basic criteria including GC content, calculated melting temperature and a sequence that is predicted to bind uniquely to a genomic locus of interest. Many other parameters can also be used in designing candidate probes to test for suitability in aCGH assays.

Methods of Producing an Array

The methods described above provide surface-bound polynucleotides with desirable binding characteristics. Once such surface-bound polynucleotides with desirable binding characteristics, i.e., “validated” surface-bound polynucleotides, have been identified, they may be used to fabricate an array for use in the subject methods. Accordingly, the invention provides a method of producing an array for performing sample-heterogeneity corrected aCGH assays. In general, the method involves identifying at least one surface-bound polynucleotide with desirable binding characteristic, and fabricating an array containing at least that polynucleotide.

In certain embodiments, a subject array may contain 1, 2, 3, more than about 5, more than about 10, more than about 20, more than about 50, more than about 100, more than about 200, more than about 500, more than about 1000, more than about 2000, more than about 5000 or more, usually up to about 10,000 or more, “validated” surface-bound polynucleotides. The arrays can also contain surface-bound polynucleotides that have not been validated.

Arrays can be fabricated using any means, including drop deposition from pulse jets or from fluid-filled tips, etc, or using photolithographic means. Either polynucleotide precursor units (such as nucleotide monomers), in the case of in situ fabrication, or previously synthesized polynucleotides (e.g., oligonucleotides, amplified cDNAs or isolated BAC, bacteriophage and plasmid clones, and the like) can be deposited. Such methods are described in detail in, for example U.S. Pat. Nos. 6,242,266, 6,232,072, 6,180,351, 6,171,797, 6,323,043, etc.

Computer-Related Embodiments

The invention also provides a variety of computer-related embodiments. Specifically, the candidate probe data analysis methods described in the previous section may be performed using a computer. Accordingly, the invention provides a computer-based system for analyzing data produced using the above methods in order to screen and identify surface-bound polynucleotides with desirable binding characteristics.

In certain embodiments, the subject methods include a step of transmitting data or results from at least one of the detecting and deriving steps, also referred to herein as evaluating, as described above, to a remote location.

Utility

The subject methods find application in, among other applications, identifying surface-bound polynucleotides, e.g., BACs, cDNAs, oligonucleotides, etc., suitable for use in sample-heterogeneity correction CGH assays. Once identified, surface-bound polynucleotides suitable for use in sample-heterogeneity correction CGH assays may be used to make a CGH array suitable for sample-heterogeneity correction processing. Accordingly, the subject methods find use in making CGH arrays.

The subject methods find use as a service provided to a user. In certain of these embodiments, the method of the present invention includes receiving an initial CGH result for a sample from a user, processing the initial CGH result to account for potential or known sample heterogeneity, and transmitting results of the processing step back to the user. In certain other of these embodiments, the methods of the present invention includes receiving a sample from a user, performing a CGH assay on the sample, processing the initial CGH result to account for potential or known sample heterogeneity, and transmitting results of the processing step to the user.

As demonstrated and discussed above, the present invention meets many current needs in the aCGH field.

Kits

Also provided by the subject invention are kits for practicing the subject methods, as described above. The subject kits at least include one probe identified using the methods above and a sample-heterogeneity reference recorded on a substrate, including paper or plastic, or in a computer readable storage medium (CD-ROM, diskette, etc.). The kits may also include calibration nucleic acid composition as described above. Other optional components of the kit include: nucleic acid labeling agents, such as for primer extension or nick translation and fluorescent labels conjugated to nucleotides. In some embodiments, arrays that comprise at least one validated probe may be included in the kits. In alternative embodiments, the kit may also contain computer-readable media for performing the subject methods, as discussed above. The various components of the kit may be present in separate containers or certain compatible components may be precombined into a single container, as desired.

In addition to above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

In addition to the subject database, programming and instructions, the kits may also include one or more control analyte mixtures, e.g., two or more control analytes for use in testing the kit.

As is evident from the description above, the subject invention provides the means to generate a database of aCGH profiles on any number of mixed cell populations. This database provides a means for accurately determining if an aCGH test sample contains a sub-population of “abnormal” cells, and also provides a means for identifying the specific genomic copy number variation present. In certain embodiments, a result from an aCGH assay is communicated to a vendor who compares it to a database containing the known aCGH profiles. The results of such a search are then communicated back to the originator, thereby enhancing their aCGH data analysis.

Furthermore, the present invention allows for the identification of oligonucleotide features that are particularly suited to function in aCGH assays on mixed cell populations. By testing candidate probes in aCGH assays with known reference samples and known test samples (e.g., different graded mixtures of normal and abnormal cells), specific probes can be identified that demonstrate a linear correlation with the amount of abnormal cells present.

As such, the subject invention provides a number of benefits and features, and represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

The following examples are offered by way of illustration and not by way of limitation.

### EXAMPLES

Exemplary Computational Methods of the Invention

A matrix (P) is constructed using the pure cell type signatures (called the matrix of pure signatures) in which the columns of P represent the genome copy number (GCN) measurements of the normal and abnormal cells studied for a selected set of probes. The columns represent a unit quantity of the relevant cell-type. The set of probes used in the analysis may be all probes represented on the array, or a subset thereof, defined on the basis of the quality of the measurement, chromosomal location, or any other parameter of interest.

Let q be a vector of quantities, representing the number of each cell-type present in a measured sample. Let m be the GCN profile measured for the sample. Note that P and m are measured ratios or signal intensities, but not logarithmic ratios. Assuming linearity of GCN measurements, one arrives at Equation 1:
Pq=m (Equation 1)
As m is measurable, Equation 1 is solved to get an estimate of unknown q.

In some cases, the equation may not be directly solvable. In these cases, a least square solution is computed. Explicitly, to compute an estimate of the vector of quantities q representing a sample with GCN profile m one uses:
q*=LSQ[Pq=m]=(P^{T}P)⁻¹P^{T}m, (Equation 2)
where LSQ denotes the least square solution for the over-determined system of linear equations (Equation 1) (Golub, G. H. and Van Laon, C. F. (1989) Matrix Computations, Baltimore Md.: Johns Hopkins University Press).

In some cases, the a priori measurements are done on mixed samples of known composition, rather than on pure samples. In fact, such measurements can provide a more robust estimate of the matrix P.

Let R be a matrix whose columns represent the known compositions of the measured samples, and let M be the matrix of the corresponding profiles. Then, by linearity:
PR=M (Equation 3)
We now solve for P. In the case when R is an invertible matrix:
P=MR⁻¹ (Equation 4)
If R is not invertible (as will be the case when redundant measurements are used):
P=MR^{T}(RR^{T})⁻¹ (Equation 5)

In order to obtain a stable solution in Equation 3, R should be designed to have a small condition number (Golub, G. H. and Van Laon, C. F. (1989) Matrix Computations, Baltimore Md.: Johns Hopkins University Press).

In the example of Table 2 (see below), R is 2 by 7 matrix with entries:

| | | | | | | |
|---|---|---|---|---|---|---|
| 0 | 0.1 | 0.3 | 0.5 | 0.7 | 0.9 | 1 |
| 1 | 0.9 | 0.7 | 0.5 | 0.3 | 0.1 | 0 |

Assuming CGH design spanning 40,000 loci (e.g. a microarray with 40,000 features), the unknown matrix P is a matrix of size 40,000 by 2, and the results of microarray measurements are expressed as 40,000 by 7 matrix M.

In another variant of the computational methods of the invention, the proportions of one or more components of the mixture are known a priori. For example the concentration of normal cell populations such as infiltrating lymphocytes and stroma in a tumor biopsy can be determined by staining methods (e.g. hematoxylin and eosin) used in standard histology and pathology analyses. In that case, Equation 1 is rewritten as:
P_{U}q_{U}=m−P_{K}q_{K}, (Equation 6)
where Pᵤ is the part of matrix P corresponding to GCN profiles of components of the mixture with unknown concentrations, qᵤ is the vector of unknown concentrations, Pₖ is the part of matrix P corresponding to GCN profiles of components of the mixture with known concentrations, and qₖ is the vector of known concentrations.

In a variant of the invention, P, M and m are the results of the preprocessing of the raw GCN data. Said preprocessing produces a step-function along the genome where each step represents a measured aberration. For example, such preprocessing is described in (P. Hupe, N. Stransky, J. P. Thiery, F. Radvanyi, and E. Barillot. Analysis of array CGH data: from signal ratio to gain and loss of DNA regions. Bioinformatics, 20(18):3413-22, 2004, Doron Lipson, Yonatan Aumann, Amir Ben-Dor, Nathan Linial, Zohar Yakhini, “Efficient Calculation of Interval Scores for DNA Copy Number Data Analysis”. Proceedings of RECOMB '05, LNCS 3500, p. 83, Springer-Verlag, 2005).

Exemplary Cell Mixtures

The effect of normal cell mixtures on aCGH measurements are modeled using samples containing variable amounts of two known cell populations (for example as shown in Table 1). To mimic an in vivo sample, two cell populations, one containing genomic lesion(s) (population 1) and the other a normal diploid genome (population 2) are mixed prior to DNA extraction, and then processed for aCGH hybridization using a reference sample (for example, derived from 46,XX).

In example (A), cell population 1 is a diploid cell line derived from a male (46,XY) patient with one intact and one truncated copy of chromosome 18 (18q-). The breakpoint on 18q21.3 in this cell line (GM50122) has been cloned and sequenced to the exact nucleotide position. The second cell population consists of a normal diploid female (46, XX) cell line with two intact copies of chromosome 18. Target nucleic acids are generated from mixtures as indicated in Table 1 and aCGH hybridizations are performed to measure the effect of diploid cell admixture on the detection of single copy intra-chromosomal (18q) and chromosomal (X chromosome) copy number alterations.

In example (B), cell population 1 is the aneuploid colon carcinoma cell line HT-29, which contains a series of well know regions of copy number gains (whole chromosomes and intra-chromosomal amplicons of variable sizes) and losses (single copy and double copy deletions) throughout its genome. Target nucleic acids are generated from mixtures as indicated in Table 1 and aCGH hybridizations are performed.

TABLE 1

| | |
|---|---|
| Cell Population 1 | Cell population 2 |

| (A) 46, XY, 18q- (%) | (B) HT-29 (%) | 46, XX (%) |
|---|---|---|
| 0 | 0 | 100 |
| 10 | 10 | 90 |
| 30 | 30 | 70 |
| 50 | 50 | 50 |
| 70 | 70 | 30 |
| 100 | 100 | 0 |

In another example, the effect of mixed neoplastic clonal populations is measured using the colorectal carcinoma cell line HT-29 and Colo320DM (Table 2). HT-29 cells are hypertriploid with a median number of 70 chromosomes and Colo320DM cells are hyperdiploid with median number of 50 chromosomes. Each of these cell lines has been analyzed by aCGH and contain distinct regions of losses and gains (Pollack et al., Nat. Genet. (1999) 23: 41-46; Snijders et al., Nat. Genet. (2001) 29:263-264)]. The use of these two tumor cell lines mimics the effect of a sample containing mixing aneuploid cell populations.

TABLE 2

| Cell Population 1 | Cell population 2 |
|---|---|
| HT-29 (%) | Colo320DM (%) |
| 0 | 100 |
| 10 | 90 |
| 30 | 70 |
| 50 | 50 |
| 70 | 30 |
| 90 | 10 |
| 100 | 0 |

The results from these experiments are processed using mathematical methods described above. This provides information useful in correcting CGH assay data from unknown samples as well as identifying oligonucleotide aCGH probes that behave in a linear fashion in the presence of cell mixtures (e.g. normal/tumor; tumor/tumor). With this information, a list of probes that are most tolerant to cell mixtures is generated, which list is then employed to determine probe design rules that predict these probes.

In some experiments, genomic samples from a pure (i.e., 100%) normal (i.e., diploid) cell population or a pure (i.e., 100%) abnormal cell population are used in an aCGH assay. In these embodiments, each genomic sample is tested against the same reference sample (e.g., a genomic sample from normal cells). The results from these assays provide copy number measurements of pure samples representative of the normal cell population and pure samples representative of the abnormal cell population. Using the results obtained from these aCGH assays, the mixture coefficients of specific mixtures of these cell populations are calculated, e.g., using the equations described above.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method comprising:
(a) providing an initial CGH result for a sample; and
(b) processing said initial CGH result to account for potential or known sample heterogeneity.

2. The method according to claim 1, wherein said initial CGH result is provided by performing a CGH assay on said sample.

3. The method according to claim 2, wherein said CGH assay is an array CGH assay.

4. The method according to claim 1, wherein said processing comprises comparing said result to a sample-heterogeneity reference.

5. The method according to claim 4, wherein said method comprises changing said results to account for any sample heterogeneity identified in said comparing.

6. The method according to claim 4, wherein said sample-heterogeneity reference is a collection comprising a plurality of probe results for a plurality of different sample compositions.

7. The method according to claim 6, wherein said collection is present in a database.

8. The method according to claim 1, wherein said sample is a tumor biopsy from a subject.

9. The method according to claim 1, wherein said sample comprises normal cells and abnormal cells.

10. The method according to claim 1, wherein said method is a method of diagnosing a subject for a neoplastic disease condition.

11. The method according to claim 1, wherein said method is a method of monitoring the clonal make-up of a tumor cell population over time.

12. The method according to claim 11, wherein said tumor cell population is propagated in an in vitro cell culture system.

13. The method according to claim 12, wherein said tumor cells are propagated in the presence of a therapeutic agent, wherein said therapeutic agent is known or predicted to inhibit the growth of said tumor cells.

14. The method according to claim 11, wherein said tumor cell population is present in a subject.

15. The method according to claim 14, wherein said subject is being treated with a therapeutic agent, wherein said therapeutic agent is known or predicted to inhibit the growth of said tumor cells.

16. A method of assessing a candidate CGH probe nucleic acid, said method comprising:
(a) performing a CGH assay with said candidate probe on a plurality of different sample-heterogeneity calibration compositions to obtain a plurality of CGH results for said candidate probe; and
(b) evaluating said plurality of CGH results to assess said candidate CGH probe nucleic acid.

17. The method according to claim 16, wherein at least one of said plurality of different sample-heterogeneity calibration compositions comprises a known mixture of at least two different cell types.

18. The method according to claim 17, wherein said at least two different cell types includes a normal cell type and an abnormal cell type.

19. The method according to claim 18, wherein said normal cell type has a normal genomic copy number with respect to a locus of interest and said abnormal cell type has an abnormal genomic copy number with respect to said locus of interest.

20. The method according to claim 16, wherein said plurality of different sample heterogeneity calibration compositions comprises a set of compositions in which the ratio of a first cell type to a second cell type progressively increases.

21. The method according to claim 20, wherein said ratio progressively increases in said set from about 0.1% to about 99.9%.

22. The method according to claim 16, wherein said evaluating step comprises comparing empirical results for at least some of said calibration compositions to expected results for said calibration compositions.

23. The method according to claim 22, wherein said expected results are results that have been determined using an algorithm that solves mixture coefficients for unknown compositions using copy number measurement of at least two different known compositions.

24. The method according to claim 22, wherein said expected results are results that have been determined using an algorithm that uses linear equations to jointly determine the integer values of copy number changes in predetermined regions using a single mixture coefficient.

25. The method according to claim 16, wherein said evaluating comprises comparing a signal obtained from said candidate CGH probe nucleic acid to a reference value.

26. The method of claim 16, wherein said candidate CGH probe nucleic acid is an oligonucleotide.

27. The method according to claim 16, wherein said method is a method of assaying said candidate CGH probe nucleic acid for suitability for use in array-based comparative genome hybridization assay.

28. The method of claim 27, wherein the method further comprises identifying a surface-bound nucleic acid suitable for use in array-based comparative genome hybridization assays.

29. The method according to claim 16, further comprising determining a sequence of said candidate CGH probe in silico.

30. A method of producing an array, comprising:
(a) identifying by a method according to claim 16 a CGH probe nucleic acid suitable for use in array-based comparative genome hybridization assay; and
(b) fabricating an array comprising said surface-bound polynucleotide.

31. An array of surface-bound nucleic acids, wherein at least one of said surface-bound nucleic acids has been identified using the method of claim 16.
